(19) United States
(12) Patent Application Publication
(10) Pub. No.: US 2004/0147014 A1
(43) Pub. Date: Jul. 29, 2004

(54) Chemical reaction circuit for cell-free protein synthesis

(76) Inventor: Koji Ikuta, Aichi (JP)

Correspondence Address: WENDEROTH, LIND & PONACK, L.L.P., 2033 K STREET N. W., SUITE 800, WASHINGTON, DC, 20006-1021, US

(21) Appl. No.: 10/484,167

(22) PCT Filed: Jan. 18, 2002
(86) PCT No.: PCT/JP02/00320

(30) Foreign Application Priority Data
Jul. 19, 2001 (JP) 2001-21948

(51) Int. Cl. C12M 1/40

(52) U.S. Cl. 435/293.1; 435/68.1; 435/288.5; 435/288.7; 435/297.2

(57) ABSTRACT

A chemical reaction circuit for cell-free protein synthesis characterized by, as a chemical reaction circuit for synthesizing a protein, having at least one reagent tank for storing a reagent for the protein synthesis and/or a reagent for detecting a product, a unidirectional valve for preventing the reverse-flow of the reagent(s), and a reaction tank connected to the reagent tank(s) via the unidirectional valve. This circuit is usable as a micro reactor whereby cell-free protein synthesis can be carried out.

### TECHNICAL FIELD

The present invention relates to a chemical reaction circuit for cell-free protein synthesis useful as a portable system for conducting cell-free synthesis of a protein. More specifically, the present invention relates to a chemical reaction circuit for cell-free protein synthesis enabling cell-free synthesis of a protein using a trace amount of reagent.

### BACKGROUND ART

Recently, intending high speed, high efficiency and integration of chemical experiments or super miniaturizing of analysis equipments, there are active studies on “chemical IC” or “biochemical IC” having inside a chemical reaction circuit for conducting basic chemical operations such as reaction, separation, extraction, detection and the like.

Chemical IC has various advantages as compared with conventional devices such as capability of measurement and analysis with a small amount of sample, capability of carrying, realization of low cost, capability of disposal and the like, and particularly has high usefulness in an experiment system using an expensive reagent and an operation of screening a substance. Up to now, methods of introducing various structures onto a substrate have been carried out.

For example, there are practically used methods of producing an electrode and channel on a silicon or glass substrate using a fine processing technology in the semiconductor field. Specifically, micro electrodes used in an electric chemical detector of liquid chromatography, a small electric chemical sensor in the medical site, and the like are produced using photolithography and etching technologies.

However, chemical IC obtained by methods based on such conventional photolithography and etching technologies uses silicon or glass as a substrate, therefore, when used as an analysis equipment or chemical reaction circuit, has various problems. For example, a silicon wafer is opaque, and resultantly has problems that detection by transmitted light frequently conducted in the chemical field cannot be carried out, and the wafer tends to be cracked. Production of chemical IC by these methods needs specific apparatuses and equipments and such as for working in a clean room and the like, and the resulted product is as extremely expensive as tens of thousands of yen per piece in the case of a micro electrode on a glass substrate. Therefore, though low cost, disposable use and the like are expected, use in a general chemical experiment is not realistic under actual conditions.

Further, in method of producing chemical IC in which a channel is formed by photolithography and etching technologies, formation of a complicated three dimensional structure is actually impossible, and a valve, pump, reaction vessel, condenser and the like cannot be formed on chemical IC. Further, photolithography and etching technologies are not suitable for formation of chemical IC as used in a biochemical experiment, since a high temperature treatment and chemical treatment are necessary in such technologies.

The present inventors have realized a method of irradiating light on a liquid photo-curable resin injected onto a transparent plate, to harden the photo-curable resin, as a method of producing a three dimensional fine structure with high precision (stereolithography) (“Robotics mechatronics lecture meeting lecture paper collection” pp. 545 to 546 (1992) issued by Japan Society of Mechanical Engineers, “Japan Society of Mechanical Engineers science lecture meeting” pp. 1213 to 1216 (1992) issued by Japan Society of Mechanical Engineers, Proc. of IEEE International Workshop on Micro Electro Mechanical Systems (MEMS '93), pp. 42 to 47 (1993); Japanese Patent Application Laid-Open (JP-A) No. 7-329188, and the like). Further, the present inventors have reported a hybrid micro stereolithography of incorporating inside thereof a micro part made of metal or resin such as an ultrafiltration membrane, SMA actuator and the like, and a chemical integrated circuit in the form of chemical IC having various functions such as a fine reaction vessel, condenser, micro pump, switching valve and the like formed by such as method (Proc. of IEEE International Conference on Micro Electro Mechanical Systems (MEMS '94), pp. 1 to 6 (1994); Proc. of IEEE International Conference on Micro Electro Mechanical Systems (MEMS '99), pp. 376 to 381 (1999)).

On the other hand, huge genetic information has been clarified by analysis of a genome based on human genome project and the like, and it is a new subject in this field to analyze and resolve the function of a gene. For solution of the function of a gene, study of gene expression is further paid to attention.

Gene expression is conducted by translation into a protein via a ribosome present in a cell, by mRNA transferred from DNA. Therefore, in study of gene expression, there is generally used a cell-using protein synthesis method using live cells such as E. coli, bacterium and the like. However, in a live cell, various self-defense mechanisms by the cell self act, and additionally, it is necessary, after cultivation of a protein containing an introduced gene, to break the cell and separate and purify the intended protein, therefore, there are a lot of constrains such as complicated synthesis operations and the like.

Then, a cell-free protein synthesis method is generally known for conducting protein synthesis outside of a cell. In such a method, separation and purification of a protein can be significantly simplified without being restricted by the defense mechanisms of a cell by using a cell extract of wheat germ and E. coli and adding an amino acid, energy substances such as ATP and the like, and DNA. However, in the case of such cell-free protein synthesis method, there is a problem that, when conducted in batch-mode, the reaction terminates in about 2 hours, and consequently, the amount of the resulting protein is smaller for the amount of a reagent used.

Against such a problem, Spirin et al. have suggested a method of cell-free protein synthesis (A. S. Spirin, V. I. Baranov, L. A. Ryabova, S. Y. Ovodov and Y. B. Alakhov, Science 242, 1162-1164 (1988)). In this method, the synthesis reaction can be continued for decades hours by feeding continuously an amino acid, ATP and the like necessary for synthesis. As a result, there is an advantage that the amount of the synthesized protein becomes from several times to decades times of that in the batch-mode method.

If various cell-free protein synthesis systems typified by such a continuous cell-free protein synthesis system can be miniaturized, it becomes possible to conduct a gene expression test using a small amount of reagents used in protein synthesis, and cost can be reduced, resultantly, such miniaturized systems are useful as an experiment apparatus for post genome study. Further, such systems are also expected to be applied to various fields such as order made medical care and intra-body embedded devices and the like for which limited production of diversified products is inevitable.

Then, the present invention has been accomplished in view of conditions as described above, and an object thereof is to solve the problems of conventional technologies and to provide a micro reactor enabling cell-free protein synthesis.

### DISCLOSURE OF INVENTION

For attaining the above-mentioned object, the present invention provides, in a first aspect, a chemical reaction circuit for cell-free protein synthesis comprising at least

one or more reagent storage tanks for storing a reagent for protein synthesis and/or a reagent for product detection,

a unidirectional valve for preventing reverse flow of a reagent and

a reaction tank connected to the above-mentioned reagent storage tank via the unidirectional valve, all provided on the same micro chip.

In a second aspect, the present invention provides a chemical reaction circuit for cell-free protein synthesis comprising at least

one or more reagent storage tanks for storing a reagent for protein synthesis and/or a reagent for product detection,

a unidirectional valve for preventing reverse flow of a reagent and

a reaction tank connected to the above-mentioned reagent storage tank via the unidirectional valve, provided on two or more connectable micro chips.

In a third aspect, the present invention provides a chemical reaction circuit for cell-free protein synthesis according to any of the above-mentioned inventions wherein the reagent storage tank, unidirectional valve and reaction tank are produced by a hybrid micro stereolithography.

Further, in a fourth aspect, the present invention provides a chemical reaction circuit for cell-free protein synthesis according to any of the above-mentioned inventions wherein the circuit comprises a separation device for separating the protein produced in the reaction tank and a product storage tank for storing the protein separated by the separation device.

In a fifth aspect, the present invention provides a chemical reaction circuit for cell-free protein synthesis wherein the reagent storage tank, unidirectional valve, reaction tank, a separation device and product storage tank are produced by a “hybrid micro stereolithgraphy” developed by the present inventor.

In a sixth aspect, the present invention provides a chemical reaction circuit for cell-free protein synthesis wherein the separation device is an ultrafiltration membrane.

In a seventh aspect, the present invention provides a chemical reaction circuit for cell-free protein synthesis wherein the circuit comprises a detection sensor for detecting the separated protein.

In an eighth aspect, the present invention provides a chemical reaction circuit for cell-free protein synthesis wherein the reagent storage tank, unidirectional valve, reaction tank, a separation device, product storage tank and detection sensor are produced by a hybrid micro stereolithography.

In a ninth aspect, the present invention provides a chemical reaction circuit for cell-free protein synthesis obtained by connecting, at least, a chip having a reagent storage tank a chip having a unidirectional valve, a chip having a reaction tank and a product storage tank connected to the reaction tank via an ultrafiltration membrane, and a chip having a detection sensor.

In a tenth aspect, the present invention provides a chemical reaction circuit for cell-free protein synthesis according to any of the above-mentioned inventions wherein the circuit has a connector part for connecting to an outer apparatus.

Further, in an eleventh aspect, the present invention provides a cell-free protein synthesis method comprising at least the process of reacting in a reaction tank a reaction reagent A introduced into a first reagent storage tank with a reaction reagent B introduced into a second reagent storage tank, in a chemical reaction circuit for cell-free protein synthesis according to any of the first to tenth inventions, and in a twelfth aspect, the present invention provides the above-mentioned cell-free protein synthesis method wherein the reaction reagent A contains at least an amino acid, ATP, GTP and DNA and the reaction reagent B contains at least a cell extract.

The present invention provides, in a thirteenth aspect, the above-mentioned cell-free protein synthesis method wherein the protein produced in the reaction tank is separated further by a separation device.

The present invention provides, in a fourteenth aspect, the above-mentioned cell-free protein synthesis method wherein the protein separated and purified by the separation device is detected and confirmed by a detection sensor, and in a fifteenth aspect, the above-mentioned cell-free protein synthesis method wherein the detection and confirmation of the protein is conducted by feeding a protein detection reagent introduced into a third storage tank to the product storage tank.

The present invention provides, in a sixteenth aspect, a cell-free protein synthesis method comprising the processes of

introducing a reaction reagent A containing at least an amino acid, ATP, GTP and DNA into a first reagent storage tank,

introducing a reaction reagent B containing at least a cell extract into a second reagent storage tank,

feeding the reaction reagent A and reaction reagent B into a reaction tank to cause reaction thereof,

further feeding the reaction reagent A into the reaction tank and

separating the produced protein by an ultrafiltration membrane,

in a chemical reaction circuit for cell-free protein synthesis according to any of the above-mentioned inventions.

Further, the present invention provides, in a seventeenth aspect, a cell-free protein synthesis method wherein the produced protein, after permeation through the ultrafiltration membrane, is stored in a product storage tank, and a protein detection reagent introduced into a third storage tank is fed to the product storage tank, and the protein is detected and conformed by a detection sensor.

Also, the present invention provides, in an eighteenth aspect, a cell-free protein synthesis method wherein feeding of a reagent is conducted by a liquid feeding device connected to a connector part, and in a nineteenth aspect, a cell-free protein synthesis method wherein the liquid feeding device is a pump.

### BRIEF DESCRIPTION OF DRAWINGS

FIG. 1 is a schematic view exemplifying a hybrid micro stereolithography of producing chemical IC of a chemical reaction circuit for cell-free protein synthesis according to this invention.

FIG. 2 is a schematic view exemplifying the structure of a chemical reaction circuit for cell-free protein synthesis according to this invention (a: birds-eye view, b: sectional view).

FIG. 3 is a view showing each chemical IC of a chemical reaction circuit for cell-free protein synthesis constituted in an example according to this invention.

FIG. 4 is a schematic view illustrating an experiment operation in conducting cell-free protein synthesis using a chemical reaction circuit for cell-free protein synthesis constituted in an example according to this invention.

FIG. 5 is a view showing the change by time of an emitting reaction in conducting luciferase synthesis using a chemical reaction circuit for cell-free protein synthesis constituted in an example according to this invention.

Marks in the figures have meanings as described below.

1: reagent storage tank

1′: reservoir chip

11: first reagent storage tank

12: second reagent storage tank

13: third reagent storage tank

2: unidirectional valve

2′: valve chip

21: first unidirectional valve

22: second unidirectional valve

23: third unidirectional valve

3′: reactor chip

31: reaction tank

32: product storage tank

4: ultrafiltration membrane

5: detection sensor, photo sensor

5′: sensor chip

6: connector part

6′: connector chip

61: first connector

62: second connector

63: third connector

64: connector for waste liquid

7: holder unit

8: liquid feeding device, pump, roller pump

9: control system, analysis apparatus

100: light

110: transparent frame

111: liquid photo-curable resin

112: hollow part

113: members such as ultrafiltration membrane and the like

### BEST MODE FOR CARRYING OUT THE INVENTION

The chemical reaction circuit for cell-free protein synthesis according to the present invention comprises at least

(a) one or more reagent storage tanks for storing a reagent for protein synthesis and/or a reagent for product detection,

(b) a unidirectional valve for preventing reverse flow of a reagent and

(c) a reaction tank connected to the above-mentioned reagent storage tank via the unidirectional valve,

provided on a micro chip. Such a chemical reaction circuit for cell-free protein synthesis may advantageously have the above-mentioned parts (a) to (c), and specific configuration, form, size and material thereof are not particularly restricted. These parts (a) to (c) may be formed on the same micro chip, or may be formed on separate micro chips. For example, a first micro chip having a reagent storage tank and a unidirectional valve and a second micro chip having a reaction tank may be connected.

The chemical reaction circuit for cell-free protein synthesis according to the present invention may further comprise

(d) a separation device for separating the produced protein, and

(e) a product storage tank for storing the protein separated by the separation device. These parts (d) and (e) may be formed, like the parts (a) to (c), on the same micro chip or on a plurality of micro chips and connected to constituted a chemical reaction circuit for cell-free protein synthesis.

In this case, exemplified as the separation device are various means such as electrophoresis, gel column, filter paper, ultrafiltration membrane and the like. Of them, the ultrafiltration membrane is preferable since the kind of the membrane can be appropriately selected depending on the separation range, and embedding into a micro chip is also relatively easy.

The chemical reaction circuit for cell-free protein synthesis according to the present invention may further comprise

(f) a detection sensor for detecting the separated protein. Such a detection sensor may also be formed and placed on the same micro chip as for the parts (a) to (e), or may be formed on separate micro chips.

As described above, the chemical reaction circuit for cell-free protein synthesis according to the present invention may be formed in any configuration, and can be designed, process and formed by conventionally known semiconductor fine processing technologies and the like. Preferably, the parts (a) to (f) are three dimensionally-formed by a hybrid micro stereolithography developed by the inventors as described above.

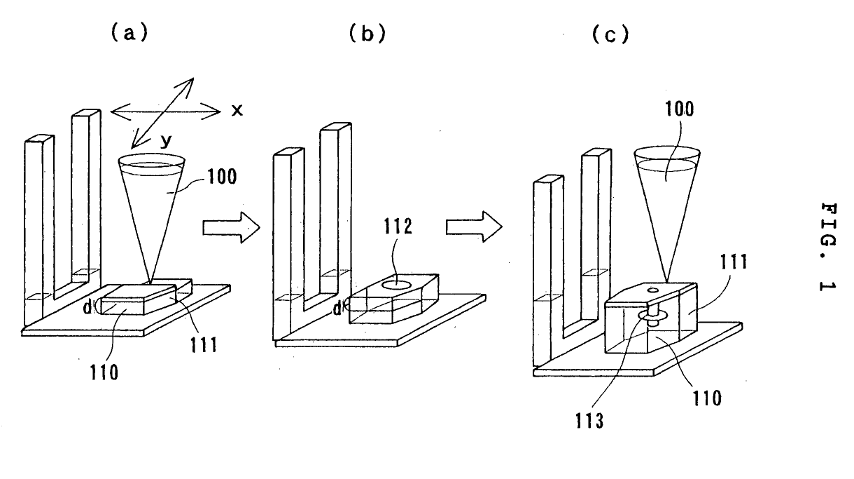

In the hybrid micro stereolithography, a liquid photo-curing polymer is injected into a frame and irradiated with light to solidified polymers at the irradiated part, forming a three-dimensional structure. Specifically, first, data designing a given steric form by a 3D-CAD system is cut by a computer at horizontal planes with constant interval along the height direction, to give a date group of the sliced graphics, and the lowest date of this sliced graphic data group is read out, and the liquid surface of a liquid photo-curable resin (111) defined in a transparent frame (110) such as glass and the like is irradiated with light (100) such as laser beam and the like while moving the light toward X and Y directions along this sliced shape, as shown in FIG. 1(a), to form a resin plate in the form of flat plate having a height of d having a shape corresponding to the above-mentioned sliced graphics, as a first layer. Next, as shown in FIG. 1(b), the transparent frame (110) was placed on the hardened resin plate as the first layer, and the liquid photo-curable resin (111) was injected to give a thickness of d, and the resin is hardened while moving light (100) along the shape of the sliced graphic date of a second layer in the same manner as described above, to harden the resin. In this operation, regarding a hollow part (112) to be made hollow, the remaining liquid photo-curable resin may advantageously be washed with a solvent and the like. By repeating the above-mentioned operation, a molded article having a given steric shape is obtained (c). In this case, it is also possible, by placing a member such as an ultrafiltration membrane (113) or the like in the liquid photo-curable resin (111) before hardening of the resin, to mold a three-dimensional structure having a complicated function such as a condenser, valve and the like (JP-A No. 7-329188).

Namely, it is preferable that the material of the main part of the chemical reaction circuit for cell-free protein synthesis according to the present invention is a photo-curable resin. Such a photo-curable resin is not particularly restricted, and there are exemplified a polyester acrylate, polyurethane acrylate, novolak type epoxy resin, bisphenol type epoxy resin and the like. In these photo-curable resins, acetophenone, benzoyl, benzyl ketal or ketone-based initiators may be added as a photo-initiator. In view of detection of a protein synthesized in this chemical reaction circuit for cell-free protein synthesis by various optical detection methods, it is preferable that the photo-curable resin has high light permeability.

Of course, in the chemical reaction circuit for cell-free protein synthesis according to the present invention, each three-dimensional structure may be formed not only by the hybrid micro stereolithography as described above but also by known or novel shaping methods such as improved methods of the hybrid micro stereolithography, and the like.

The three-dimensional structures formed by the method as described above may be sterically-formed on the same chip to constitute a chemical reaction circuit for cell-free protein synthesis according to the present invention with one micro chip, or chips having various three-dimensional structures may be combined to constitute a chemical reaction circuit for cell-free protein synthesis according to the present invention.

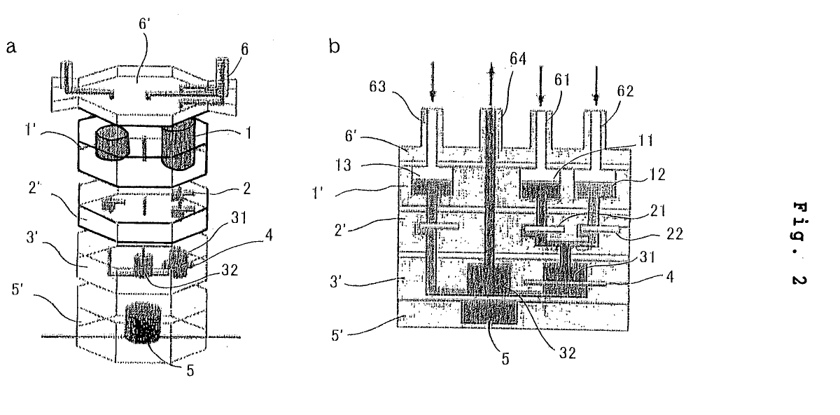

FIG. 2 shows a chemical reaction circuit for cell-free protein synthesis according to the present invention, and its constitution will be illustrated. Namely, in one example of the chemical reaction circuit for cell-free protein synthesis of the present invention shown in FIG. 2, a reservoir chip (1) having a reagent storage tank (1) for storing a reagent for protein synthesis and/or a reagent for product detection, a valve chip (2′) having a unidirectional valve (2), a reactor chip (3′) having a reaction tank (31) and a product storage tank (32) connected to the reaction tank at the down flow side via an ultrafiltration membrane (4) as a separation device, and a sensor chip (5) having a detection sensor (5), are connected to constitute the circuit.

It is preferable that such as chemical reaction circuit for cell-free protein synthesis has a connector chip (6′) having a connector part (6) for connecting to a liquid feeding device (8) for feeding each reagent to each storage tank and/or reactor.

Further, when these chips are laminated sequentially and connected and integrated by a holder unit (9), a chemical reaction circuit for cell-free protein synthesis is obtained.

Of course, the chemical reaction circuit for cell-free protein synthesis according to the present invention is not limited to that exemplified in FIG. 2, and may be constituted as one chip on which the reagent storage tank (1), unidirectional valve (2), reaction tank (31), product storage tank (32) connected to the reaction tank at the down stream side via the ultrafiltration membrane (4), and detection sensor (5) and the like are formed. When the above-mentioned hybrid micro stereolithography as reported by the present inventors is applied, such an integrated type chemical reaction circuit for cell-free protein synthesis can be formed.

In the chemical reaction circuit for cell-free protein synthesis as described above, it size is not particularly restricted. However, in view of use as a micro reactor, it is preferable that the circuit is finer in the range in which opening and closing of a valve, liquid flow and reaction are not disturbed. For example, it is possible that the thickness of each chip is 5 mm or less, and the height of a chemical reaction circuit for cell-free protein synthesis is less than 3 cm. Of course, it may be smaller or larger depending on applications.

The invention of the instant application also provides a method of synthesizing a protein using the chemical reaction circuit for cell-free protein synthesis as described above and without receiving an influence of the self defense functions of a cell. Specifically exemplified a method of continuously synthesizing a protein by introducing a reaction reagent A containing at least an amino acid, ATP, GTP and DNA into a first reagent storage tank (11), introducing a reaction reagent B containing at least a cell extract into a second reagent storage tank (12), feeding the reaction reagent A and reaction reagent B into a reaction tank (31) having an ultrafiltration membrane (4) to case reaction thereof, further, feeding the reaction reagent A to the reaction tank (31).

The protein produced in the operation is separated by the ultrafiltration membrane (4), and stored in a product storage tank (32) connected to the down stream side via the ultrafiltration membrane (4). By feeding a protein detection reagent introduced into a third storage tank (13) to this product storage tank (32), the protein produced can be detected by a detection sensor (5).

As described above, though the configuration and the like of the above-mentioned parts are not particularly restricted, it is preferable, since the product storage tank (32) acts also as a detection part, that the tank is placed in parallel to the detection sensor (5), and is positioned at the center part of the horizontal section of the chemical reaction circuit for cell-free protein synthesis.

Of course, the cell-free protein synthesis method according to the present invention may be advantageously be conducted using the chemical reaction circuit for cell-free protein synthesis as described above, and its synthesis procedure and conditions are not limited to those described above. Namely, if the number of constituent parts, configuration, size and the like of the chemical reaction circuit for cell-free protein synthesis are appropriately designed and formed, conventionally known or novel various synthesis methods can be applied.

In the cell-free protein synthesis method as described above, it is preferable that feeding of a reagent is conducted by a liquid feeding device (8) connected to connector parts (61, 62, 63), and as this liquid feeding device (8), various pumps are exemplified. Reagents and the like after reaction may be discarded out of the chemical reaction circuit for cell-free protein synthesis from a connector for waste liquid (64), or fed again from connector parts (61, 62, 63) and re-used.

Further, feeding of each reagent is conducted by controlling opening closing of each unidirectional valve (2). Therefore, in the chemical reaction circuit for cell-free protein synthesis according to the present invention, a controlling system (9) for conducting opening and closing of a valve (2) and on/off of a pump (8), further, analysis and data analysis in a sensor (5), and the like, and wirings for this system may be provided.

Of course, the protein to be synthesized in the chemical reaction circuit for cell-free protein synthesis according to the present invention as described above is not particularly restricted. By optionally altering conditions such as the kind and number of the reagent, the constitution of the reaction tank, temperature and the like, various proteins containing an enzyme can be synthesized.

Examples will be shown below referring to appended figures, and embodiments of the present invention will be illustrated further in detail below. Of course, it is needless to say that the present invention is not limited to the following examples, and various embodiments in detailed portions are possible.

### EXAMPLES

### Example 1

### Construction of Chemical Reaction Circuit for Cell-Free Protein Synthesis

According to the hybrid micro stereolithography suggested by the present inventors (JP-A Nos. 7-329188, 2001-158050, 2001-157662, 2001-158000 and the like), a connector chip (6) as a micro chip having a connector part (6), a reservoir chip (1′) as a micro chip having a reagent storage tank (1), a valve chip (2′) as a micro chip having a unidirectional valve (2), a reactor chip (3′) as a micro chip having a reaction tank (31) and a product storage tank (32), and a sensor chip (5′) as a micro chip having a photo sensor (5), were shaped. A silicon membrane was incorporated into the valve chip (2′), an ultrafiltration membrane (MICROCON YM100, manufactured by Miropore) was integrated into the reactor chip (3′), and an avalanche photo diode was integrated into the sensor chip (5). Each micro chip was made as an octagon of 14 mm and a thickness of 2 to 5 mm excepting the connector chip (6). Further, the diameter of a flow route was 300 μm.

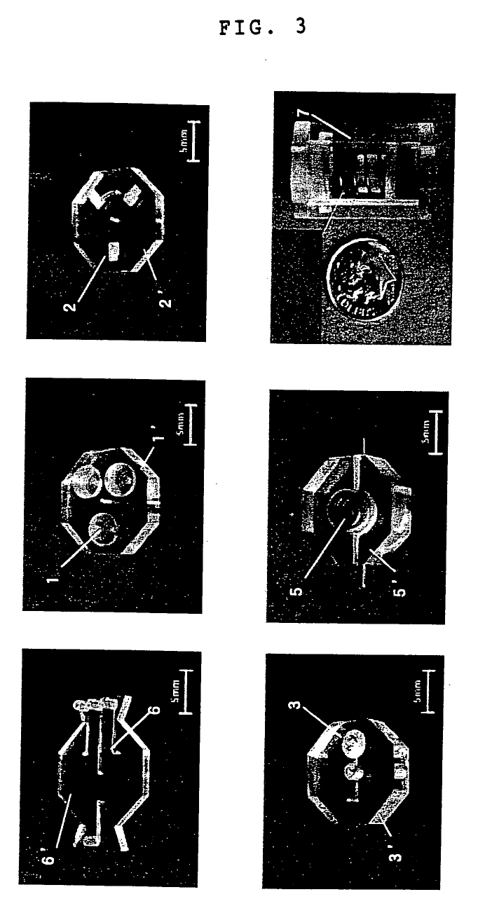

FIG. 3 shows the shapes of the micro chips (1′ to 6′) and a holder unit (7) for connecting these micro chips in lamination. The sectional view of the chemical reaction circuit for cell-free protein synthesis obtained by connecting the micro chips is as shown in FIG. 2(b).

### Example 2

### Protein Synthesis Test

A test of protein synthesis was conducted using the chemical reaction circuit for cell-free protein synthesis as constructed in Example 1. The intended protein was firefly luminescence enzyme luciferase detectable easily.

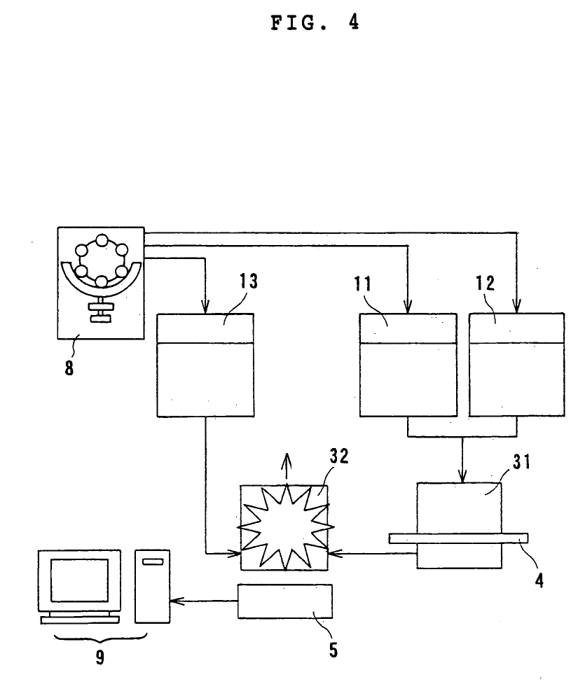

FIG. 4 shows a schematic view of a chemical reaction circuit system for cell-free protein synthesis used for synthesis of luciferase.

(a) The feeding connectors (51, 52, 53) on the connector chip (5) of the chemical reaction circuit for cell-free protein synthesis as constructed in Example 1 were connected to the discharge side of a roller pump (8), and a discharge connector (54) was connected to the waste liquid tank (8). Into a first storage tank (11) on the reservoir chip (1′) was introduced a reagent A (containing amino acid, ATP, GTP, DNA) composed of 3 μl of ultra pure water, 1.25 μl of Amino Acid Mixture 1 (manufactured by Promega, L1020 E. coli S30 Extract System for Circular DNA kit), 1.25 μl of Amino Acid Mixture 2 (manufactured by Promega, L1020 E. coli S30 Extract System for Circular DNA kit), 10 μl of Premix without Amino Acids (manufactured by Promega, L1020 E. coli S30 Extract System for Circular DNA kit) and 2 μl or less of pBEST/ucDNA, Circular (1 μg/μl; manufactured by Promega, L1020 E. coli S30 Extract System for Circular DNA kit), into a second storage tank (12) was introduced a reagent B composed of 7.5 μl of S30 Extract, Circular (manufactured by Promega, L1020 E. coli S30 Extract System for Circular DNA kit), and into a third storage tank (13) was introduced Luciferin (manufactured by Promega, Luciferase Assay Reagent) as a luciferin detection reagent.

(b) Moving the roller pump (8), the reagent A was fed to the reaction tank (31) through the first unidirectional valve (21), and the reagent B was fed to the reaction tank (31) through the second unidirectional valve (22). The reaction tank was kept at 35° C. for incubation for 1 hour.

(c) Further, the first unidirectional valve (21) was opened, and the reagent A was fed to the reaction tank (31).

(d) Luciferase synthesized in the reaction tank (31) permeated through the ultrafiltration membrane (4) of the reaction tank (31), and was fed to the detection part at the down stream (product storage tank) (32).

(e) The third unidirectional valve (23) was opened, luciferin was fed to the product storage tank (32), and the emission intensity was measured by the photo sensor (5) and analyzed by the analysis apparatus (9).

(f) Incubation at 35° C. was conducted for 1 hour, to continue the synthesis reaction. Further, the processes (d) to (e) were repeated every one hour.

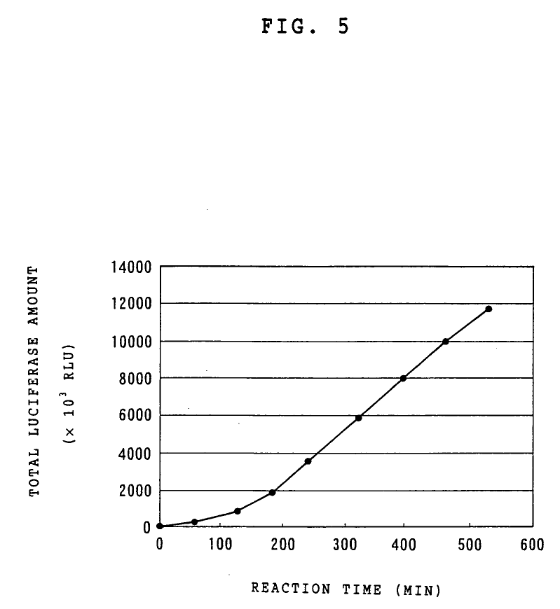

The results of measurement of the emission reaction by an enzymatic reaction using luciferin and ATP as a substrate are shown in FIG. 5.

From FIG. 5, it is shown that synthesis of luciferase was continued over 8 hours or more, and the total synthesis amount of luciferase increased with the reaction time.

By this, it was confirmed that it is possible to synthesize a protein in a micro system using a chemical reaction circuit for cell-free protein synthesis according to the present invention.

### INDUSTRIAL APPLICABILITY

As described in detail above, a micro reactor enabling cell-free protein synthesis is provided by the present invention. Such a chemical reaction circuit for cell-free protein synthesis is useful for synthesis of a protein in a cell-free system using a trace amount of reagent, and can be appropriately designed depending on the intended protein and protein synthesis methods. Further, not only batch-wide synthesis of a protein in a cell-free system, but also continuous synthesis becomes possible.

Therefore, the chemical reaction circuit for cell-free protein synthesis of the present invention can be used for a gene expression test using a small amount of reagent used in protein synthesis, and the like, and useful as an experiment apparatus for post genome study. Further, an application thereof to order made medical care and intra-body embedded devices and the like for which limited production of diversified products is inevitable is also expected.

1. A chemical reaction circuit for cell-free protein synthesis comprising at least
one or more reagent storage tanks for storing a reagent for protein synthesis and/or a reagent for product detection,
a unidirectional valve for preventing reverse flow of a reagent and
a reaction tank connected to said reagent storage tank via the unidirectional valve, all provided on the same micro chip.

2. A chemical reaction circuit for cell-free protein synthesis comprising at least
one or more reagent storage tanks for storing a reagent for protein synthesis and/or a reagent for product detection,
a unidirectional valve for preventing reverse flow of a reagent and
a reaction tank connected to said reagent storage tank via the unidirectional valve, provided on two or more connectable micro chips.

3. The chemical reaction circuit for cell-free protein synthesis according to claim 1 or 2, wherein the reagent storage tank, unidirectional valve and reaction tank are produced by a hybrid micro stereolithography.

4. The chemical reaction circuit for cell-free protein synthesis according to claim 1 or 2, wherein the circuit comprises a separation device for separating the protein produced in the reaction tank and a product storage tank for storing the protein separated by the separation device.

5. The chemical reaction circuit for cell-free protein synthesis according to claim 4, wherein the reagent storage tank, unidirectional valve, reaction tank, a separation device and product storage tank are produced by a hybrid micro stereolithography.

6. The chemical reaction circuit for cell-free protein synthesis according to claim 4 or 5, wherein the separation device is an ultrafiltration membrane.

7. The chemical reaction circuit for cell-free protein synthesis according to claim 4, wherein the circuit comprises a detection sensor for detecting the separated protein.

8. The chemical reaction circuit for cell-free protein synthesis according to claim 7, wherein the reagent storage tank, unidirectional valve, reaction tank, a separation device, product storage tank and detection sensor are produced by a hybrid micro stereolithography.

9. A chemical reaction circuit for cell-free protein synthesis obtained by connecting, at least,
a chip having a reagent storage tank, a chip having a unidirectional valve,
a chip having a reaction tank and a product storage tank connected to the reaction tank via an ultrafiltration membrane, and
a chip having a detection sensor.

10. The chemical reaction circuit for cell-free protein synthesis according to any of claims 1 to 9, wherein the circuit has a connector part for connecting to an outer apparatus.

11. A cell-free protein synthesis method comprising at least the process of reacting in a reaction tank a reaction reagent A introduced into a first reagent storage tank with a reaction reagent B introduced into a second reagent storage tank, in the chemical reaction circuit for cell-free protein synthesis according to any of claims 1 to 10.

12. The cell-free protein synthesis method according to claim 11, wherein the reaction reagent A contains at least an amino acid, ATP, GTP and DNA and the reaction reagent B contains at least a cell extract.

13. The cell-free protein synthesis method according to claim 11 or 12, comprising the process of further separating the protein produced in the reaction tank by a separation device.

14. The cell-free protein synthesis method according to claim 13, wherein the protein separated and purified by the separation device is detected and confirmed by a detection sensor.

15. The cell-free protein synthesis method according to claim 14, wherein the detection and confirmation of the protein is conducted by feeding a protein detection reagent introduced into a third storage tank to the product storage tank.

16. A cell-free protein synthesis method comprising the processes of:
introducing a reaction reagent A containing at least an amino acid, ATP, GTP and DNA into a first reagent storage tank;
introducing a reaction reagent B containing at least a cell extract into a second reagent storage tank;
feeding the reaction reagent A and reaction reagent B into a reaction tank to cause reaction thereof;
further feeding the reaction reagent A into the reaction tank; and
separating the produced protein by an ultrafiltration membrane,
in the chemical reaction circuit for cell-free protein synthesis according to any of claims 1 to 10.

17. The cell-free protein synthesis method according to claim 16, wherein the produced protein, after permeation through the ultrafiltration membrane, is stored in a product storage tank, and a protein detection reagent introduced into a third storage tank is fed to the product storage tank, and the protein is detected and conformed by a detection sensor.

18. The cell-free protein synthesis method according to claim 16 or 17, wherein feeding of a reagent is conducted by a liquid feeding device connected to a connector part.

19. The cell-free protein synthesis method according to claim 18, wherein the liquid feeding device is a pump.
